(19) United States
(12) Patent Application Publication
(10) Pub. No.: US 2026/0160055 A1
(43) Pub. Date: Jun. 11, 2026

(54) URINAL WITH OPENING/CLOSING LID

(71) Applicant: jaeho KIM, Gimhae-si (KR)

(72) Inventor: jaeho KIM, Gimhae-si (KR)

(21) Appl. No.: 19/179,352

(22) Filed: Apr. 15, 2025

(30) Foreign Application Priority Data
Apr. 22, 2024 (KR) 10-2024-0053317

(51) Int. Cl. E03D 13/00; A61L 2/10

(52) U.S. Cl. CPC E03D 13/005; A61L 2/10; A61L 2202/11; A61L 2202/121

(57) ABSTRACT

The present invention relates to a urinal with opening/closing lid, and exhibits the effect of preventing the diffusion of a bad odor and enabling more sanitary use through an opening/closing lid structure provided at the inlet part of the urinal body. The urinal includes: a urinal body installed at a fixed height on a wall surface; an opening/closing lid configured to be connected to an upper inlet part of the urinal body in an openable/closeable manner; an operating switch 30 protrusively provided on the inlet part of the urinal body so as to be pressed against the opening/closing lid; a water spray nozzle provided on the opening/closing lid so that water spraying can be performed by the operation of the operating switch; and a drain pipe configured to be connected to a lower part of the urinal body so that water sprayed through the water spray nozzle can be drained.

### TECHNICAL FIELD

The present invention relates to a urinal, and more particularly, to a urinal structure that is equipped with an opening/closing lid on an upper part of the urinal, thereby allowing for more convenient and sanitary use.

### BACKGROUND ART

Generally, it is a well-known fact that men's restrooms in buildings such as rest areas, parks, and amusement parks as well as various public facilities such as theaters, department stores, and subways are equipped with urinal facilities to easily urinate in a standing posture.

Such a urinal is composed of a main body having an opened front face, a cleaning water supply pipe installed in an upper part of the main body, a drain pipe installed in a lower part of the main body for discharging defecation and cleaning water, and a control unit for detecting the user's defecation and supplying a fixed amount of cleaning water.

Therefore, after defecation, the control unit opens the cleaning water supply pipe to supply a fixed amount of cleaning water into the main body, which is then discharged through the drain pipe together with the defecation, thereby keeping the inside of the main body clean.

However, despite this configuration, the prior arts have a structure in which the urinal is always opened, which causes inconveniences such as the generation of bad odor and urine splashing into the surrounding area.

### PRIOR ART LITERATURE

### Patent Literature

(Patent Literature 0001) Korean Patent No. 1253981 (registered on Apr. 8, 2013)

(Patent Literature 0002) Korean Patent No. 1752086 (registered on Jun. 22, 2017)

### DETAILED DESCRIPTION OF THE INVENTION

### Technical Problem

The present invention has been proposed to improve the problems in the above-mentioned prior arts, and the purpose thereof is to provide a urinal that is equipped with an openable and closable lid at the inlet part to prevent the generation of bad odor and prevent urine from splashing, thereby enabling more sanitary use of the urinal.

### Technical Solution

In order to achieve the above object, according to the present invention, there is provided a urinal with an opening/closing lid, comprising: a urinal body installed at a fixed height on a wall surface; an opening/closing lid configured to be connected to an upper inlet part of the urinal body in an openable/closeable manner; an operating switch protrusively provided on the inlet part of the urinal body so as to be pressed against the opening/closing lid; a water spray nozzle provided on the opening/closing lid so that water spraying can be performed by the operation of the operating switch; and a drain pipe configured to be connected to a lower part of the urinal body so that water sprayed through the water spray nozzle can be drained.

In some embodiments, the urinal body gradually increases in width as it goes from the lower part to the upper part, and the upper inlet part has a shape inclined at a prescribed angle.

In some embodiments, the urinal body is configured to enable height adjustment through a guide rail formed on a wall surface.

### Advantageous Effects

The urinal according to the present invention exhibits the effect of preventing the diffusion of a bad odor and enabling more sanitary use through an opening/closing lid structure provided at the inlet part of the urinal body.

In addition, since the cleaning water is sprayed under the state where the opening/closing lid is closed, it shows the advantage of improving the cleaning efficiency.

### BRIEF DESCRIPTION OF THE DRAWINGS

FIG. 1 is a cross-sectional structural diagram of an installation state of a urinal according to an embodiment of the present invention.

FIG. 2 is a state diagram of the process closing the opening/closing lid of the urinal of the present invention.

FIG. 3 is an enlarged view of a section A of FIG. 2.

FIG. 4 is a state diagram of the operation of closing the opening/closing lid of the urinal of the present invention.

FIG. 5 is a front view of the opening/closing lid of the urinal of the present invention in a state where the lid is opened.

FIG. 6 is a front view of the opening/closing lid of the urinal of the present invention in a state where the lid is closed.

FIG. 7 is a front structural diagram of a urinal according to another embodiment of the present invention.

FIG. 8 is a cross-sectional structural diagram of a urinal according to another embodiment of the present invention.

### DETAILED DESCRIPTION OF THE EMBODIMENTS

Hereinafter, specific embodiments of the present invention will be described in detail with reference to the accompanying drawings.

The present invention may be variously modified and have several exemplary embodiments. The present invention should not be construed as being limited to specific embodiments described in detail below. The present embodiments are provided to more completely explain the present invention to those having average knowledge in the art.

Therefore, the shapes of the components shown in the drawings may be exaggerated to emphasize a clearer explanation. It should be noted that in each drawing, the same components are sometimes depicted by the same reference numerals. In addition, detailed descriptions of the functions and configurations of known technologies that are deemed to unnecessarily obscure the gist of the present invention may be omitted.

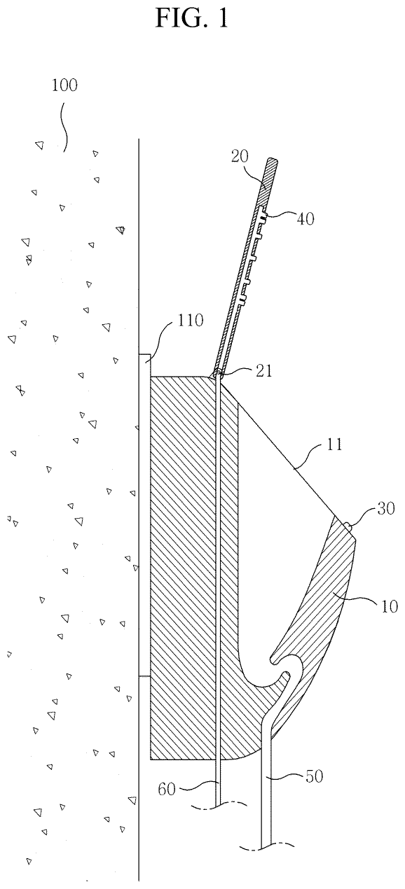

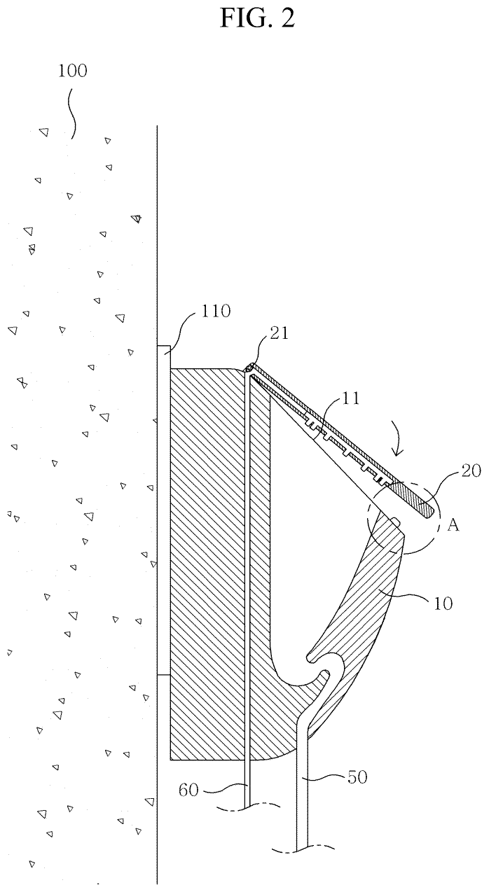

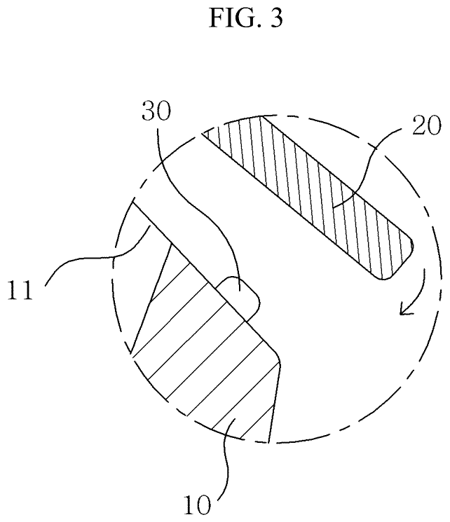

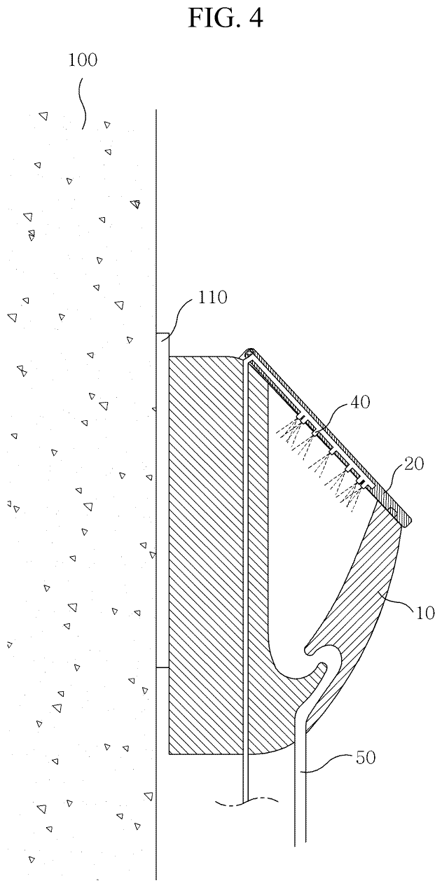

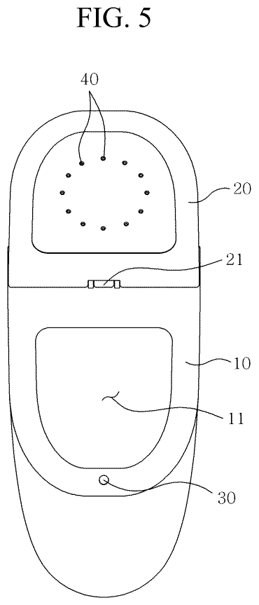

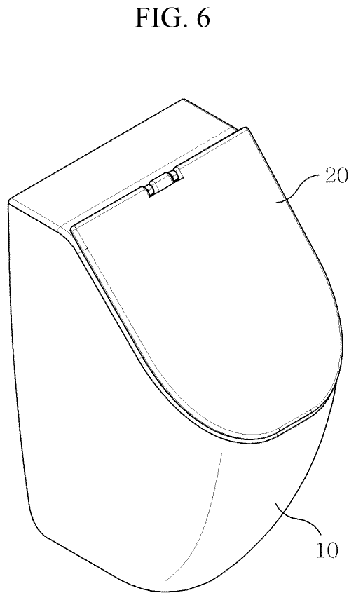

First, the structure of a urinal with an opening/closing lid according to an embodiment of the present invention is described below with reference to FIGS. 1 to 6.

A urinal according to the present embodiment comprises a urinal body 10 installed at a fixed height on a toilet wall surface 100, an opening/closing lid 20 configured to be connected to an upper inlet part of the urinal body 10 via a hinge shaft 21 in an openable/closeable manner, an operating switch 30 protrusively provided on the inlet part of the urinal body 10 so as to be pressed against the opening/closing lid 20, a plurality of water spray nozzles 40 provided on the opening/closing lid 20 so that water spraying can be performed by a control unit signal during the pressure operation of the operating switch 30; and a drain pipe 50 configured to be connected to a lower part of the urinal body 10 so that water sprayed through the water spray nozzle 40 can be drained.

At this time, the urinal body 10 gradually increases in width as it goes from the lower part to the upper part, and the upper inlet part 11 has a shape inclined at a prescribed angle.

In addition, it can be confirmed that the water spray nozzles 40 are provided in a plurality of numbers at regular intervals along the circumference of the opening/closing lid 20.

Furthermore, it is preferable that the urinal body 10 is configured to enable height adjustment through a guide rail 110 formed on a wall surface 100.

In the drawing, the unexplained symbol 60 indicates a cleaning water supply pipe through which cleaning water is supplied.

The functional effects due to the use of the urinal of the present invention having such a configuration will be considered below.

The urinal of the present invention can be fixedly installed on a wall 100, or can be installed so as to enable the lifting and lowering height adjustment through a guide rail 110.

When a user urinates at the urinal under such an installation state, the urine is discharged through the drain pipe 50.

At this time, the urinal body 10 of the present invention can prevent a phenomenon in which urine splashes or flows to the surrounding area because the upper inlet part 11 is inclined at a certain angle.

After that, when the user who finished urinating closes the opening/closing lid 20, the operation switch 30 is pressed and at the same time, a water spray operation is performed according to the control signal of the control unit. At this time, the cleaning water supplied through the cleaning water supply pipe 60 is sprayed through the water spray nozzle 40 to perform the cleaning operation.

In particular, since the opening/closing lid 20 of the present invention is provided with a plurality of water spray nozzles 40 at regular intervals, cleaning with a cleaning water can be effectively performed without the occurrence of blind spots.

In addition, the opening/closing lid 20 can be kept in a closed state, thereby preventing a phenomenon in which the bad smell generated inside the urinal is diffusing into the space inside the restroom, and at the same time improving the appearance due to the opening/closing lid 20 to perform an interior function of the restroom.

Therefore, the urinal of the present invention exhibits the effect of preventing the diffusion of bad smell and enabling more sanitary use through the opening/closing lid structure provided at the inlet part of the urinal body.

Further, since the cleaning water is sprayed under the state where the opening/closing lid is closed, which offers the advantage of improving the cleaning efficiency.

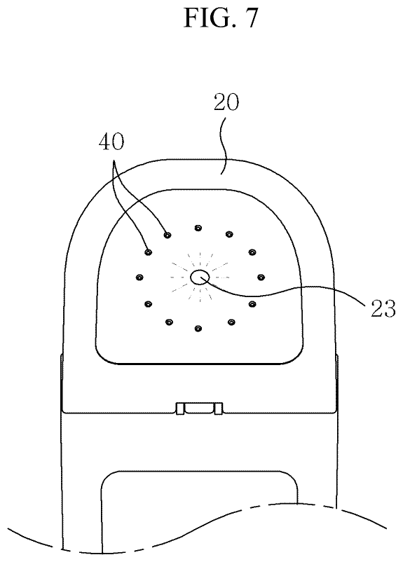

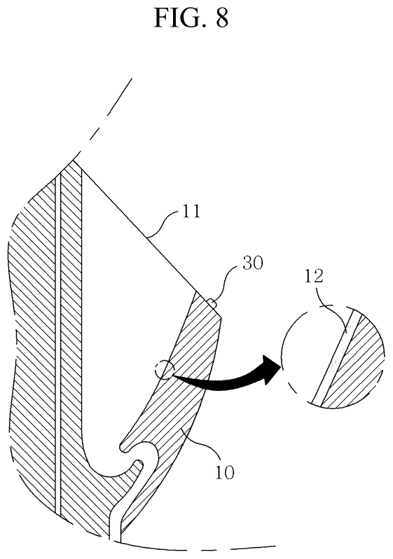

Meanwhile, FIGS. 7 and 8 show configurations of the urinal according to another embodiment of the present invention. The opening/closing lid 20 is equipped with a UV lamp 23 that generates UV light for a certain period of time when the lid is closed, and the inner wall surface of the urinal body 10 is formed to be coated with a pollution preventive layer 12 for preventing pollution due to foreign matter and performing sterilization.

At this time, the pollution preventive layer 12 is preferably formed in the mixing ratio of: 30 to 40% by weight of polypropylene resin, 10 to 20% by weight of glyceryl stearate, 5 to 20% by weight of fluorinated xenon, 1 to 10% by weight of aluminum nitride powder, 1 to 15% by weight of phenylalanine-4-hydroxylase, 1 to 10% by weight of polybenzimidazene, 1 to 10% by weight of lithium hydroxide, 1 to 5% by weight of isoflavonoid, and 1 to 10% by weight of polycaprolactone.

By achieving such a constitution, after the closing operation of the opening/closing lid 20, the inside of the urinal body 10 that has been cleaned with water is irradiated with a UV light generated from a UV lamp 23 for a certain period of time, thereby exhibiting a sterilization action on the inside of the urinal.

Furthermore, since the urinal body 10 has a pollution preventive layer 12 formed by coating on the surface, it prevents the occurrence of water stains and also prevents surface discoloration caused by urine.

In particular, since glyceryl stearate and fluorinated xenon components are incorporated into the pollution preventive layer 12, the surface lubricity is improved and the surface adhesion of foreign substances is prevented. The aluminum nitride powder improves the sterilization function due to the light reflection of UV light, and the phenylalanine tetrahydroxylase suppresses the generation of bubbles during the coating process of the pollution preventive layer 12, so that a uniform coating layer can be formed as a whole, and the polybenzimidazene and lithium hydroxide perform the function of preventing surface discoloration and pollution. In addition, the further added isoflavonoid and polycaprolactone each perform a catalytic function for the polypropylene resin and increase the bonding force between the compositions, thereby exhibiting the advantage of strengthening the coating density.

Further, although specific embodiments of the present invention have been described and illustrated above, it would be obvious that the urinal structure of the present invention can be modified and practiced in various ways by those skilled in the art.

For example, in the above embodiments, the urinal body is described and illustrated as having a structure that enables the lifting and lowering height adjustment, but it can be installed as a fixed type structure that is fixed to the wall if necessary.

In addition, the opening and closing operation of the opening/closing lid can be selectively performed in a manual or automatic manner.

If the urinal configured as above is adopted in an apartment or the like equipped with two or more urinals, the amount of water used is less than half that of toilet bowl, which offers the advantage of reducing the amount of water used.

Therefore, such modified embodiments should not be understood individually from the technical idea or scope of the present invention, and such modified embodiments should be construed to fall under the scope of the present invention as set forth in the appended claims.

### Description of Symbols

10: urinal body 11: inlet part
20: opening/closing lid 30: hinge shaft
30: operating switch 40: water spray nozzle
50: drain pipe 60: cleaning water supply pipe
100: wall 110: guide rail

What is claimed is:

1. A urinal with an opening/closing lid, comprising:
a urinal body installed at a fixed height on a wall surface;
an opening/closing lid configured to be connected to an upper inlet part of the urinal body in an openable/closeable manner;
an operating switch protrusively provided on the inlet part of the urinal body so as to be pressed against the opening/closing lid;
a water spray nozzle provided on the opening/closing lid so that water spraying can be performed by the operation of the operating switch; and
a drain pipe configured to be connected to a lower part of the urinal body so that water sprayed through the water spray nozzle can be drained,
wherein the opening/closing lid is equipped with a UV lamp that generates UV light for a certain period of time during the closing operation,
wherein the inner wall surface of the urinal body is formed to be coated with a pollution preventive layer for preventing pollution due to foreign matter and performing sterilization, and
wherein the pollution preventive layer is formed in the mixing ratio of: 30 to 40% by weight of polypropylene resin, 10 to 20% by weight of glyceryl stearate, 5 to 20% by weight of fluorinated xenon, 1 to 10% by weight of aluminum nitride powder, 1 to 15% by weight of phenylalanine-4-hydroxylase, 1 to 10% by weight of polybenzimidazene, 1 to 10% by weight of lithium hydroxide, 1 to 5% by weight of isoflavonoid, and 1 to 10% by weight of polycaprolactone.

2. The urinal with an opening/closing lid according to claim 1, wherein the urinal body gradually increases in width as it goes from the lower part to the upper part, and the upper inlet part has a shape inclined at a prescribed angle.

3. The urinal with an opening/closing lid according to claim 1, wherein the water spray nozzles are provided in a plurality of numbers at regular intervals along the circumference of the opening/closing lid.

4. The urinal with an opening/closing lid according to claim 1, wherein the urinal body is configured to enable height adjustment through a guide rail formed on a wall surface.
